(19) United States
(12) Patent Application Publication
(10) Pub. No.: US 2008/0064960 A1
(43) Pub. Date: Mar. 13, 2008

(54) HIGH FREQUENCY ULTRASOUND TRANSDUCER HOLDER AND ADJUSTABLE FLUID INTERFACE

(76) Inventors: Willet F. Whitmore III, Longboat Key, FL (US); Roger F. Wilson, Sarasota, FL (US)

Correspondence Address: STEPTOE & JOHNSON LLP, 1330 CONNECTICUT AVENUE, N.W., WASHINGTON, DC, 20036, US

(21) Appl. No.: 11/746,619

(22) Filed: May 9, 2007

Related U.S. Application Data
(60) Provisional application No. 60/746,887, filed on May 9, 2006.

(51) Int. Cl. A61B 8/00

(52) U.S. Cl. 600/459

(57) ABSTRACT

An ultrasound transducer positioning system includes a curvilinear articulating arm, an ultrasound transducer, and a holder having a first region for receiving the transducer and a second region for a fluid interface disposed adjacent the first region. The holder is coupled to the curvilinear articulating arm and the second region is adjustable in size.

### CROSS-REFERENCE TO RELATED APPLICATION

The benefits of Provisional Application No. 60/746,887 filed May 9, 2006 and entitled “High Frequency Ultrasound Transducer Holder and Adjustable Fluid Interface” are claimed under 35 U.S.C. §119(e), and the entire contents of this provisional application are expressly incorporated herein by reference thereto.

### FIELD OF THE INVENTION

The invention relates to a high frequency ultrasound transducer holder and adjustable fluid interface.

### BACKGROUND OF THE INVENTION

High frequency ultrasound (HIFU) is a recognized method of delivering treatment energy to subcutaneous lesions in the body. Most of the transducers that have been developed for this purpose have focal zones that are at a fixed distance from the face of the transducer where the sound waves are focused to achieve the energy concentration for treatment. This feature requires that either the transducer or the patient be moved for the treatment area to be shaped to a treatment volume as desired. In addition, some fluid medium must be present between the patient and the transducer that allows the ultrasound energy to be efficiently transmitted. Typically this fluid medium has been degassed water held within a flexible container. Since it is highly desirable to prevent immersion of the HIFU transducer and to be able to adjust the depth of the focal zone in relation to the patient, treatment tables for supporting the patient, the fluid interface and the transducer have been developed for these purposes. There remains a need for an improved HIFU transducer holder.

### SUMMARY OF THE INVENTION

The invention relates to an ultrasound transducer positioning system including a curvilinear articulating arm, an ultrasound transducer, and a holder comprising a first region for receiving the transducer and a second region for a fluid interface disposed adjacent the first region, the holder being coupled to the curvilinear articulating arm. The second region is adjustable in size.

The first region may be defined by a generally circular clamp which may be a split ring. A lead screw may be coupled to the clamp. The second region may be defined between the clamp and an opposing members and the lead screw may extend to the opposing member. The opposing member may be a ring. The transducer may be retained by at least one member coupled to the clamp and abutting a portion of the transducer. In some embodiments, the at least one member may abut a portion of a face of the transducer. The fluid interface may be a flexible member. The system may further include at least one rail with indexing thereon, the rail extending between a bracket coupled to the curvilinear articulating arm and a clamp defining the first region.

The present invention is designed to provide a portable, self contained, adjustable fluid interface and HIFU transducer holder that will perform the required functions for accurate and effective treatment and keep the treatment field and transducer dry. This design obviates the need for a specialized table to support the transducer and fluid interface and to move the transducer and patient in a controlled relationship.

Among the advantageous features of the present invention are: it is readily attachable to a supporting positioning arm that has six degrees of freedom for initial positioning of the device; it has a receptacle for securely grasping the transducer; it has a generally cylindrical cage for supporting and aiding in the shaping and positioning of a disposable balloon that may be filled with a fluid interface medium such as degassed water.

The cage may be adjusted in length with both rapid and fine adjustment mechanisms which determine the distance of the transducer from the patient contacting surface of the device (thus controlling the depth of the focal zone). The balloon may be easily purged of air and is made of elastic material that causes minimal attenuation of the ultrasound energy. Depending on the position of the transducer in relation to the patient contacting surface of the device (i.e. the length of the balloon cage), the fluid volume in the balloon may be adjusted to achieve full contact of the balloon with both the entire active surface of the transducer and the patient. The end of the balloon cage opposite the transducer may have a circular lipped opening to allow for a large contact surface for the balloon with the patient. The lip may be smooth and tapered to contain the balloon in the cage while permitting desired patient contact with minimal chance for air gaps. Ultrasound gel or some equivalent may be used on the interface surfaces of the balloon where it comes in contact with the transducer and the patient to optimize energy transmission. In addition to a manual fine adjustment mechanism for the length of the cylindrical balloon cage that can account for the depth of the focal zone in the patient (Z axis), this device may also include a fine adjustment mechanism that allows manual position adjustment in the X-Y plane. This allows the operator to create a shaped treatment volume for a lesion in a patient.

### BRIEF DESCRIPTION OF THE DRAWINGS

Preferred features of the present invention are disclosed in the accompanying drawings, wherein:

FIGS. 1A-1I show a first embodiment of a high frequency ultrasound transducer holder for providing an adjustable fluid interface in accordance with the present invention including (1A) a side perspective view, (1B-1E) side views, (1F) a bottom view, (1G) a top view, (1H) a partial cross-sectional perspective view of the Z-axis adjustment mechanism, and (1I) a perspective view of a spring-loaded button of the Z-axis adjustment mechanism of FIG. 1H, with each of the figures shown without a balloon for convenience except FIG. 1B;

FIGS. 1J to 1-O show a variant of the first embodiment of a high frequency ultrasound transducer holder for providing an adjustable fluid interface in accordance with the present invention including (1J) a side perspective view, (1K-1L) side views, (1M) a top view, (1N) a partial cross-sectional perspective view of the Z-axis adjustment mechanism, and (1-O) a perspective view of a spring-loaded button of the Z-axis adjustment mechanism of FIG. 1N, with each of the figures shown without a balloon for convenience except FIG. 1K;

FIGS. 2A-2K show another embodiment of a high frequency ultrasound transducer holder for providing an adjustable fluid interface in accordance with the present invention including (2A) a side perspective view, (2B-2E) side views, (2F) a top view, (2G) a bottom view, (2H-J) side views of the center block for the positioning device for the X-Y axes, and (2K) a partial cross-sectional side view of the center block of FIGS. 2H-J, with each of the figures shown without a balloon for convenience except FIG. 2B;

FIG. 3 shows a perspective view of a high frequency ultrasound transducer holder system 200 according to the present invention;

FIGS. 4A-4C show the curvilinear articulating arm assembly of FIG. 3, including (4A) a perspective view, (4B) a partial cross-sectional perspective view, and (4C) a partial side view;

FIGS. 4D-4L show the base handle of FIG. 3, including (4D) a first side view, (4E) a second side view, (4F) a partial perspective view of a first set of components thereof, (4G) a partial side view of a second set of components thereof, (4H) another partial side view of the second set of components thereof, (4I) a front view, (4J) a back view, (4K) a top view, and (4L) a bottom view;

FIG. 4M shows a perspective view of a rail clamp for use with the present invention;

FIGS. 4N-4T show the free handle of FIG. 3, including (4N) a first side perspective view showing a portion of a tensioning wire therewith, (4-O) a second side perspective view, (4P) a partial perspective view showing a first set of components thereof, (4Q) a front perspective view, (4R) a back perspective view, (4S) a top perspective view, and (4T) a bottom perspective view;

FIG. 4U shows a side perspective view of the interface lock of the free handle of FIGS. 4N-4T;

FIG. 5 shows a perspective view of a support system according to the present invention; and

FIGS. 6A-6C show the tray of FIG. 5, including (6A) a top view, (6B) a cross-section taken perpendicular to the central axis of the tray, and (6C) a partial cross-section showing detail taken at VIC.

### DETAILED DESCRIPTION OF THE PREFERRED EMBODIMENTS

Words of orientation as used herein such as “front,” “back,” “top,” and “vertical” are used for exemplary convenience only as non-limiting examples of the orientation of features and are not intended to have any particular limiting effect.

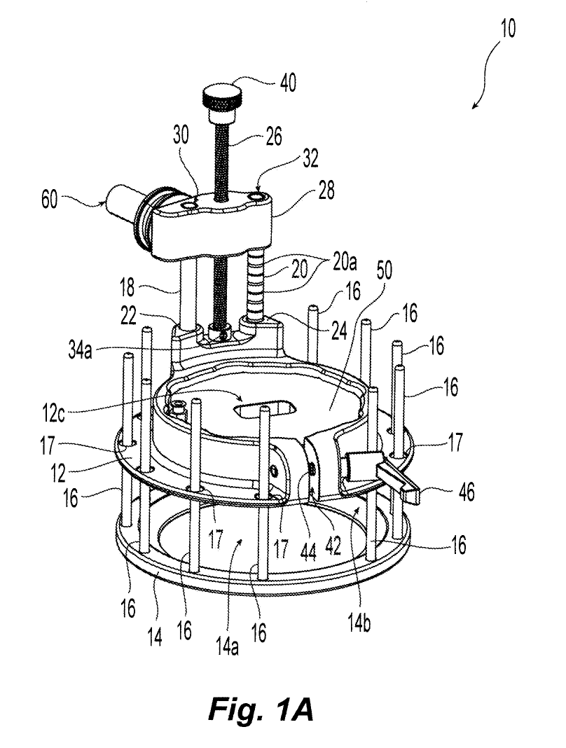

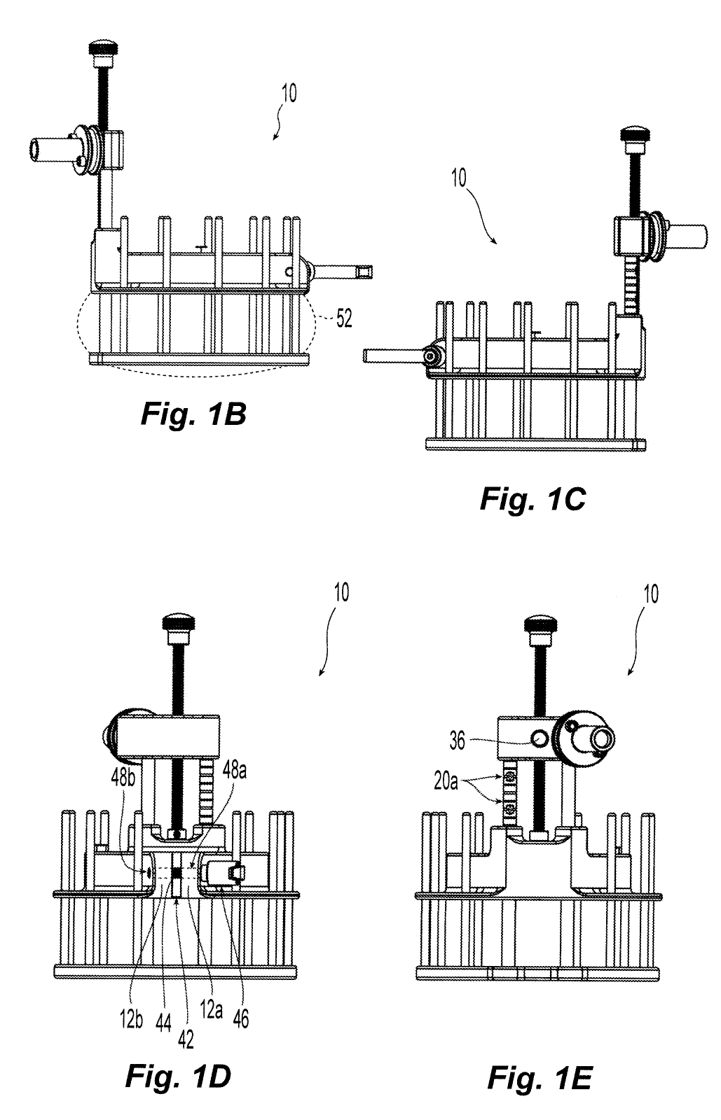

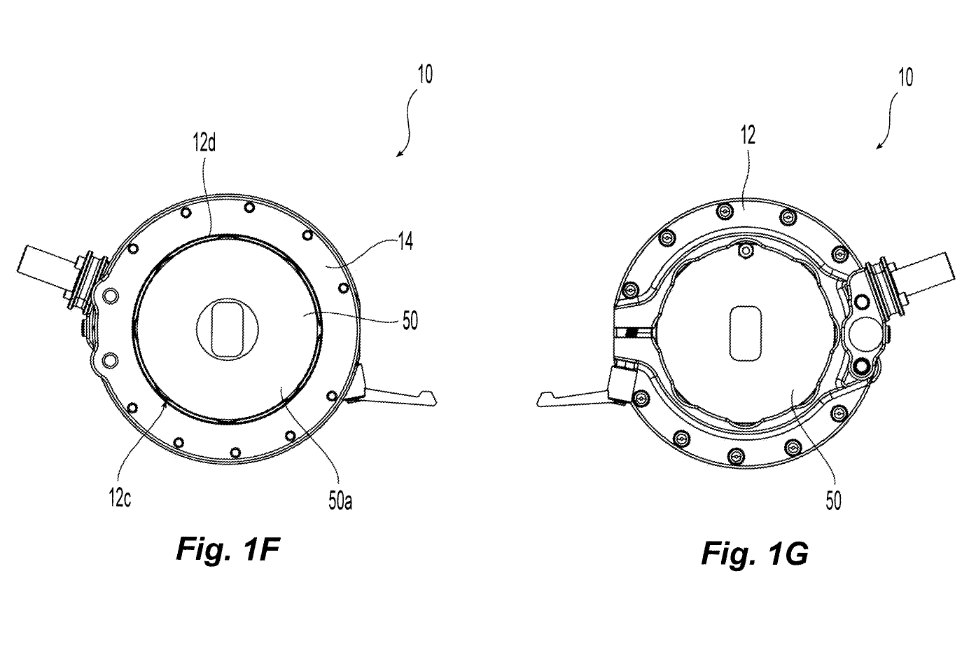

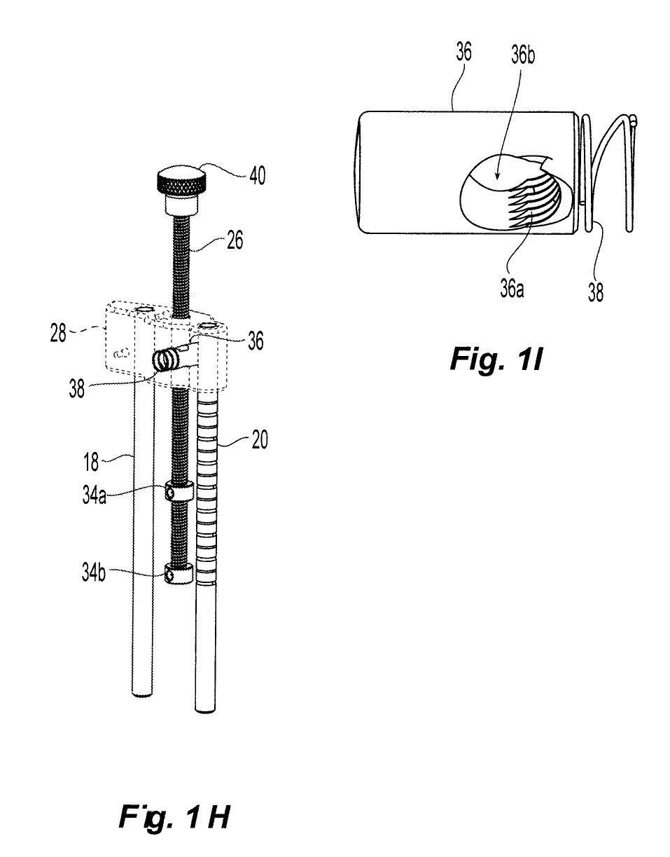

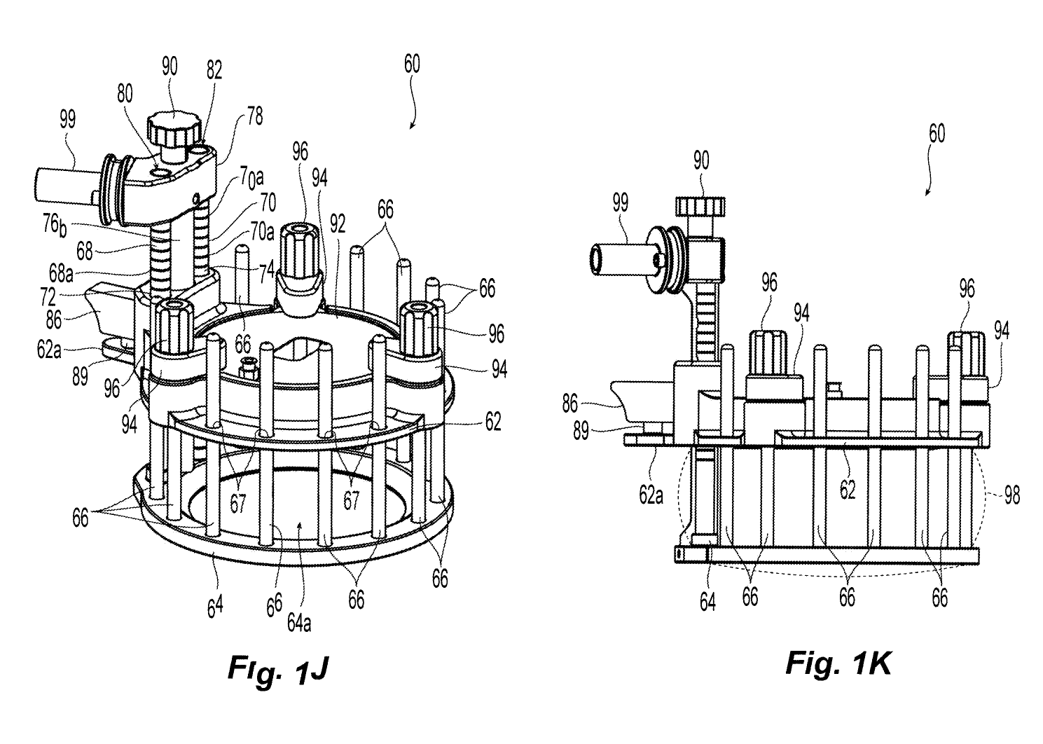

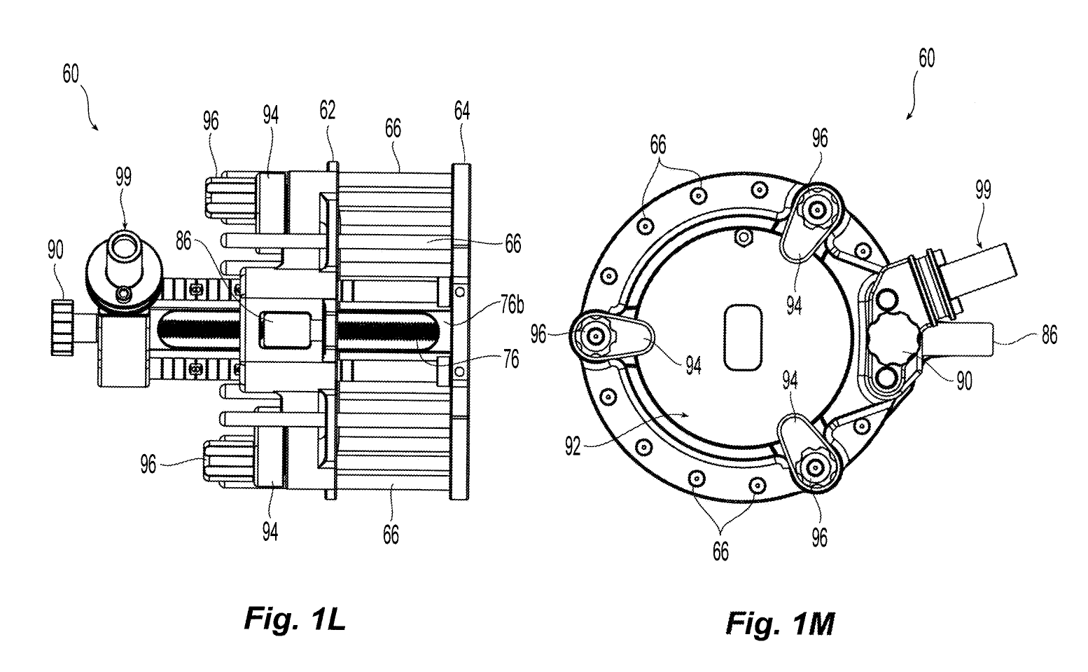

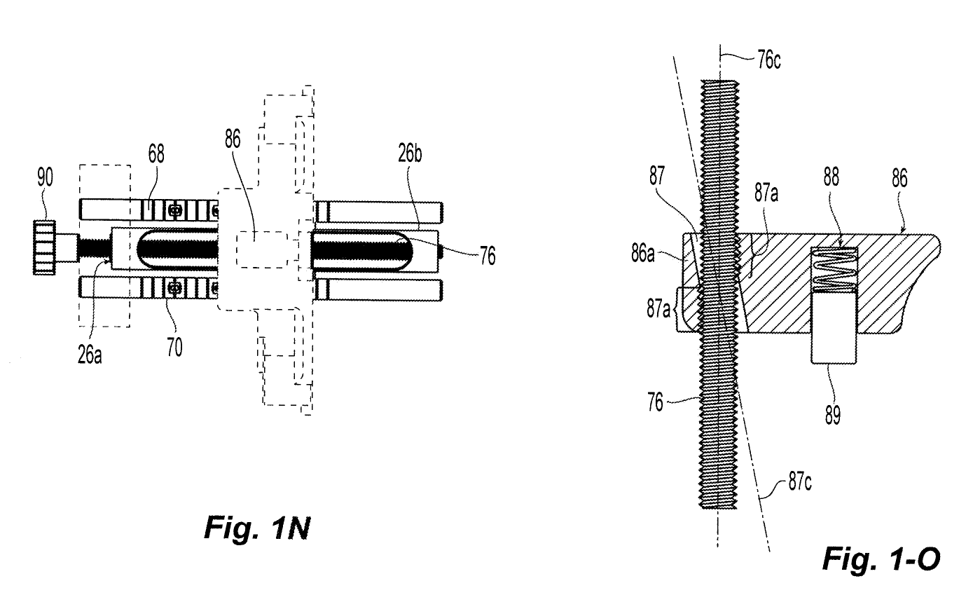

Referring initially to FIGS. 1A-1I, a first exemplary embodiment of a high frequency ultrasound transducer holder 10 for providing an adjustable fluid interface in accordance with the present invention is shown. Holder 10 includes an ultrasound probe clamp 12 and an opposing bottom ring 14. A plurality of rods 16 are fixed circumferentially about ring 14 and extend through aligned holes 17 in clamp 12. In addition, a pair of guide rails 18, 20 are fixed circumferentially about ring 14 and extend through aligned holes 22, 24, respectively, in clamp 12. Rail 20 may include indexing 20a such as scoring, grooves, indicia including numerical indicia, etc., which for example may indicate the separation distance between portions of transducer holder 10 as will be described. A lead screw 26 extends through a hole (not shown) in probe clamp 12 centrally disposed between holes 22, 24. Each of guide rails 18, 20 also are received in primary bracket 28 in holes 30, 32 respectively therein. A spaced pair of internally threaded collars are threadably associated with lead screw 26 and each abut clamp 12.

For movement of clamp 12 with respect to ring 14 in the Z-axis, both gross and fine movements may be achieved. As shown for example in FIG. 1H, a button 36 is disposed in primary bracket 28 and extends perpendicular to the Z-axis defined by or parallel to lead screw 26. Button 36 is spring-loaded with a spring 38 abutting a face of button 36 and disposed within primary bracket 28. Button 36 is partially internally threaded with partial threads 36a disposed in a hole 36b. Hole 36b is sized such that lead screw 26 extends therethrough. Due to the spring-loading provided by biasing member 38, button 36 is biased such that partial threads 36a thereof engage the threads of lead screw 26 when button 36 is not depressed. When a user depresses button 36 and compresses spring 38, hole 36b of button 36 accommodates movement of partial threads 36a away from engagement with the threads of lead screw 26 so that gross movement of clamp 12 with respect to ring 14 may be permitted in the Z-axis. Fine movement of clamp 12 with respect to ring 14 may be permitted in the Z-axis when the partial threads 36a of button 36 are engaged with the threads of lead screw 26 and a user rotates lead screw 26 about or parallel to the Z-axis by rotating lead screw 26 with knob 40. In summary, vertical adjustment screw 26 allows fine tuning of the focal zone depth associated with transducer 50, while button 36 may be depressed for making larger rapid vertical adjustments.

As shown in FIGS. 1A and 1D, clamp 12 is formed as a split ring with a gap 42 therein. A screw 44 with a handle 46 fixed to a free end thereof extends through holes 48a, 48b and gap 42 may be increased or decreased in size by rotation of screw 44, using handle 46, to move opposing portions 12a, 12b away from or toward each other. By decreasing the size of gap 42, an ultrasound transducer 50 such as a HIFU transducer, is secured within clamp 12, as shown for example in FIG. 1A. In an exemplary preferred embodiment, the outer diameter of transducer 50 closely matches the inner diameter of hole 12c of clamp 12, at a preferably predetermined size of gap 42, so that transducer 50 may be mechanically held by clamp 12. A flexible member such as a balloon 52 preferably is disposed and retained between (1) the combination of clamp 12 and bottom face 50a of transducer 50 and (2) ring 14.

As shown for example in FIG. 1B, balloon 52 is permitted to extend through hole 14a in ring 14, thus permitting this fluid reservoir to be in contact with both transducer 50 and the skin of a patient proximate ring 14. Ring 14 preferably includes a tapering 14b in thickness toward hole 14a which for example may be spherical or another arcuate cross-section. This dished configuration facilitates resting of balloon 52 against ring 14. Clamp 12 may include similar tapering 12d with respect to central hole 12c therein.

In a preferred exemplary embodiment, balloon 52 is stretchy, strong, and causes minimal attenuation of the signal from transducer 50 when gel is coated on both the transducer contact and patient contact surfaces; preferably, balloon 52 is filled with degassed water with air purged therefrom.

In a preferred exemplary embodiment, transducer 50 focuses ultrasound about 10 centimeters away from the bottom surface 50a thereof. In order to change the depth in the patient to which the energy is directed and focused, the spacing of clamp 12 and ring 14 with respect to each other is adjustable for example between about 1 centimeter and about 9 centimeters. Spacing of clamp 12 and transducer 50 retained in ring 14 for example may be adjusted with reference to numerical indicia 20a such as shown in FIG. 1E.

A coupling 60 may be used to couple holder 10 to a curvilinear articulating arm as will be described.

Referring next to FIGS. 1J-1-O, a variant exemplary embodiment of the first embodiment is shown. In particular, a high frequency ultrasound transducer holder 60 for providing an adjustable fluid interface includes an ultrasound probe clamp 62 and an opposing bottom ring 64. A plurality of rods 66 are fixed circumferentially about ring 64 and extend through aligned holes 67 in clamp 62. In addition, a pair of guide rails 68, 70 are fixed circumferentially about ring 64 and extend through aligned holes 72, 74, respectively, in clamp 62. Each of rails 68, 70 may include indexing 68a, 70a such as scoring, grooves, indicia including numerical indicia, etc, which for example may indicate the separation distance between portions of transducer holder 60 as will be described. A lead screw 76 (show in FIGS. 1L, 1N, and 1-O) extends through a hole (not shown) in probe clamp 62 centrally disposed between holes 72, 74. Each of guide rails 68, 70 also are received in primary bracket 78 in holes 80, 82 respectively therein. An internally threaded collar 76a is threadably associated with lead screw 76 and abuts a lead screw housing or guard 76b.

For movement of clamp 62 with respect to ring 64 in the Z-axis, as with the previous embodiment both gross and fine movements may be achieved. Turning for example in FIG. 1N, a button 86 is associated with clamp 62 and extends perpendicular to the Z-axis defined by or parallel to lead screw 76. Button 86 is spring-loaded, with a spring 88 abutting (1) a face of button 86 and (2) a face of a nut plunger 89 (which may for example be right cylindrical). Nut plunger 89 abuts a ledge or extension 62a of clamp 62. Button 86 also is disposed adjacent clamp 62, and abuts clamp 62 as will be described. In addition, button 86 includes a bore 87 that is partially internally threaded with threaded regions 87a, 87b disposed therein. Bore 87 is sized such that lead screw 76 extends therethrough. Bore 87 is disposed along an axis 87c transverse to the longitudinal axis 76c of lead screw 76. Advantageously, the “rocker” style of button 86 does not rely on spring force entirely to engage lead screw 76. With a balloon 98 disposed between clamp 62 and ring 64, as the spacing between clamp 62 and ring 64 is decreased the balloon 98 exerts pressure against clamp 62 with the result that button 86 is levered with increasing force of engagement with the threads of lead screw 76.

Due to the spring-loading provided by biasing member 88, button 86 is biased such that partial threads 87a, 87b thereof engage the threads of lead screw 76 when button 86 is not depressed. When a user depresses button 86 and compresses spring 88, bore 87 of button 86 accommodates movement of partial threads 87a, 87b away from engagement with the threads of lead screw 76 so that gross movement of clamp 62 with respect to ring 64 may be permitted in the Z-axis. In particular, by depressing button 86 proximate spring 88, a portion of face 86a of button 86 “rocks” against clamp 62 to permit pivoting of button 86 with respect to lead screw 76, thereby permitting disengagement of their respective threads and allowing gross movement. Conversely, gross movement may be arrested when the respective threads are engaged.

Fine movement of clamp 62 with respect to ring 64 may be permitted in the Z-axis when the partial threads 87a, 87b of button 86 are engaged with the threads of lead screw 76 and a user rotates lead screw 76 about or parallel to the E-axis by rotating lead screw 76 with knob 90. In summary, vertical adjustment screw 76 allows fine tuning of the focal zone depth associated with transducer 92, while button 86 may be depressed for making larger rapid vertical adjustments.

Unlike the first embodiment, holder 10, the exemplary variant embodiment includes a clamp 62 that preferably is not in the form of a split ring. Instead, an ultrasound transducer 92, such as a HIFU transducer, is secured within clamp 62, as shown for example in FIGS. 1J and 1M. With respect to holder 60, transducer 92 is “top-loaded” to facilitate installation of transducer 92 in clamp 62. In other words, transducer 50 is placed within clamp 62 without having to traverse the region occupied between clamp 62 and ring 64. In a preferred exemplary embodiment, paired retention washer 94 and associated retention knob 96 are used to secure transducer 92 within clamp 62. Preferably, three point retention is used, e.g., three pairs of washers 94 and associated knobs 96 are used and may be disposed for example about 120° apart. Washers 94, in the exemplary embodiment as shown, are oblong in shape and may be positioned to bear against transducer 92 threadably tightened so that when transducer 92 is seated in clamp 62, it is retained therein. In particular, knobs 96 may include threaded shafts (not shown) that are threadably received in holes in clamp 62, so that when a washer 94 is disposed between a knob 96 and clamp 62 the degree of tightness or looseness may be adjusted to permit transducer 92 to be retained in, or removed from clamp 62. When transducer 92 is to be removed from clamp 62, for example, each coupling is loosened so that washers 94 may be swiveled out of abutment with transducer 92.

As shown for example in FIG. 1K, balloon 98 is permitted to extend through hole 64a in ring 64, thus permitting this fluid reservoir to be in contact with both transducer 92 and the skin of a patient proximate ring 64. Ring 64 preferably includes a tapering and configuration as previously described with respect to the previous embodiment. Similarly, balloon 98 and transducer 92 for example may be designed and used as previously described.

Finally, a coupling 99 may be used to couple holder 10 to a curvilinear articulating arm as will be described.

Preferably, holder 60 is formed of materials so that it is substantially radiolucent and magnetic resonance imaging compatible (the use of stainless steel is kept minimal, for example by only using stainless steel screws and springs).

Advantageously, as may be seen by comparing FIGS. 1A and 1J, holder 60 of FIG. 1J is a “low profile” holder. In particular, the locking mechanism including button 86 is associated directly with clamp 62, rather than with bracket 28 as with holder 10. In addition, lead screw 76 of holder 60 is separated from the region between clamp 62 and ring 64 by a barrier, guard 76b so that balloon 98 does not contact lead screw 76. Such an arrangement ensures that balloon 98 is not compromised as a result of contacting sharp surfaces of screw 76. In sum, holder 10 includes a lead screw 26 which does not extend to ring 14. Because no guard is provided in that exemplary embodiment, lead screw 26 was positioned away from the region between clamp 12 and ring 14, and as a result the overall height of holder 10 included much of the length screw 26 extending beyond clamp 12. In contrast, because lead screw 76 of holder 60 extends to ring 64, the same length of a lead screw 76 can be accommodated with a decrease in overall height of holder 60 compared to holder 10. Of course, the separation distance between a transducer in clamp 12, 62 and respective ring 14, 64 is a major factor in setting the depth of the focus of the ultrasound.

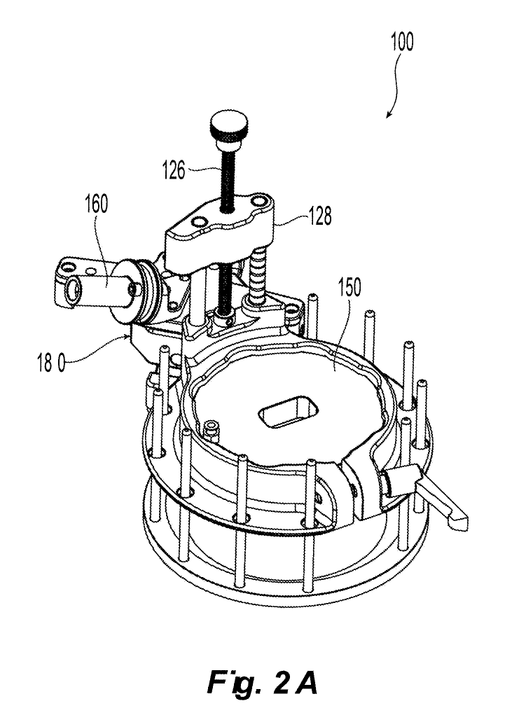

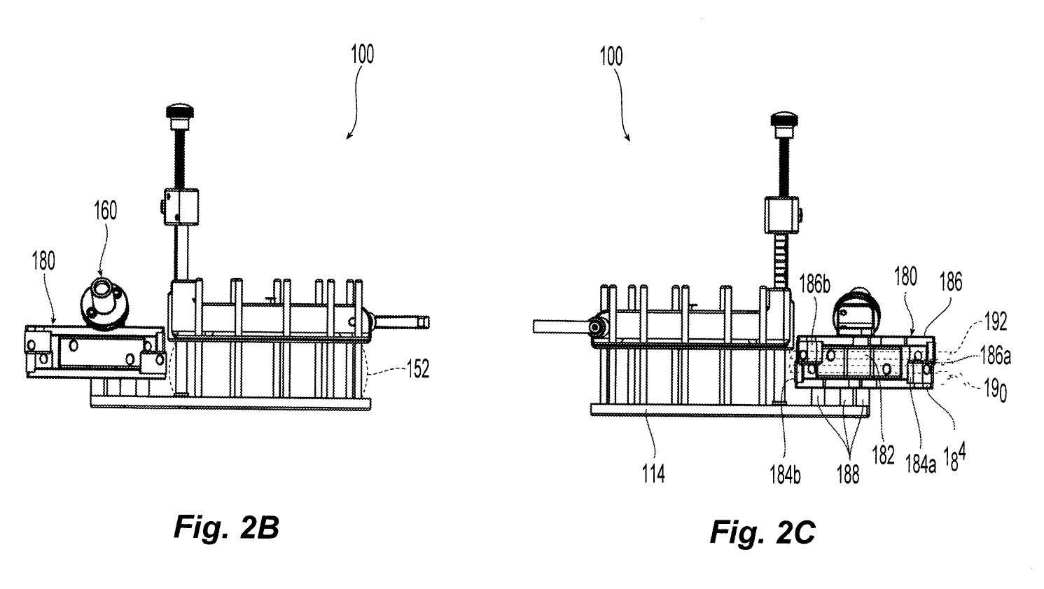

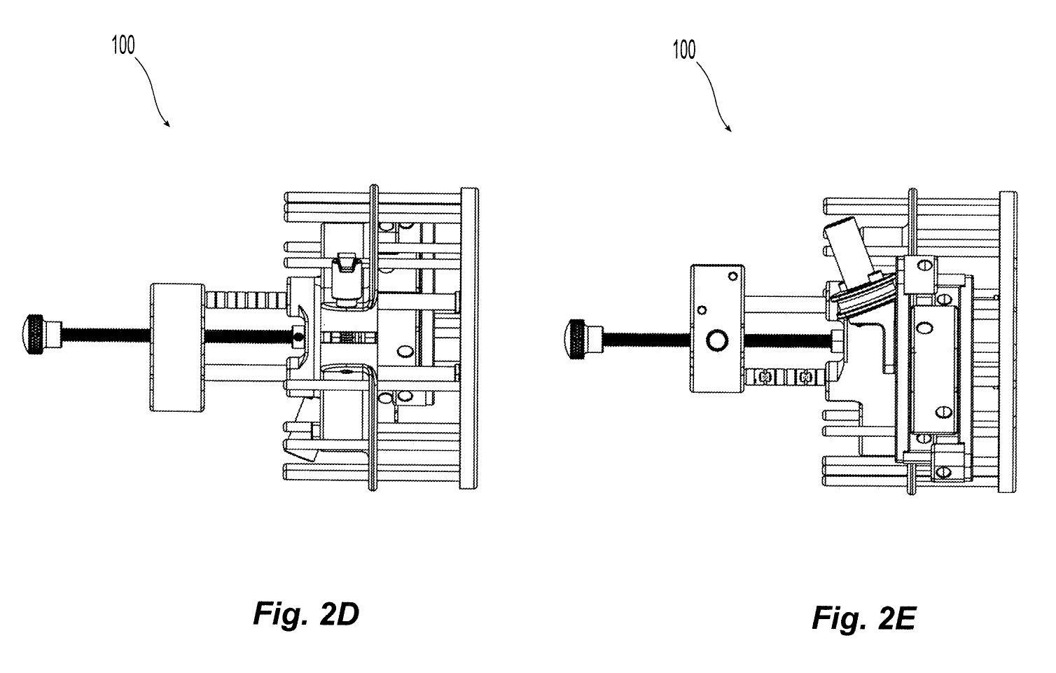

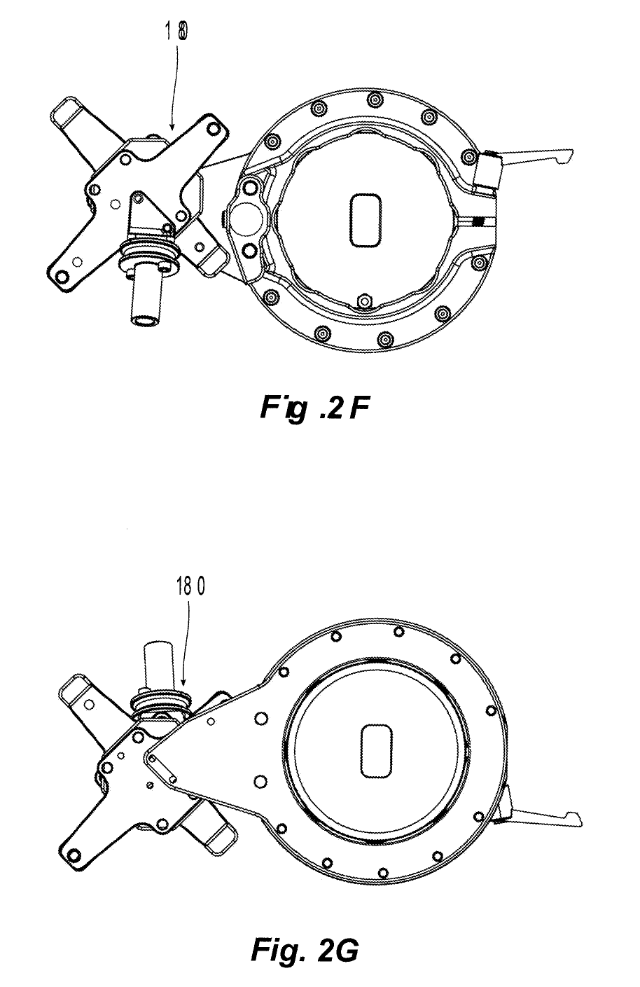

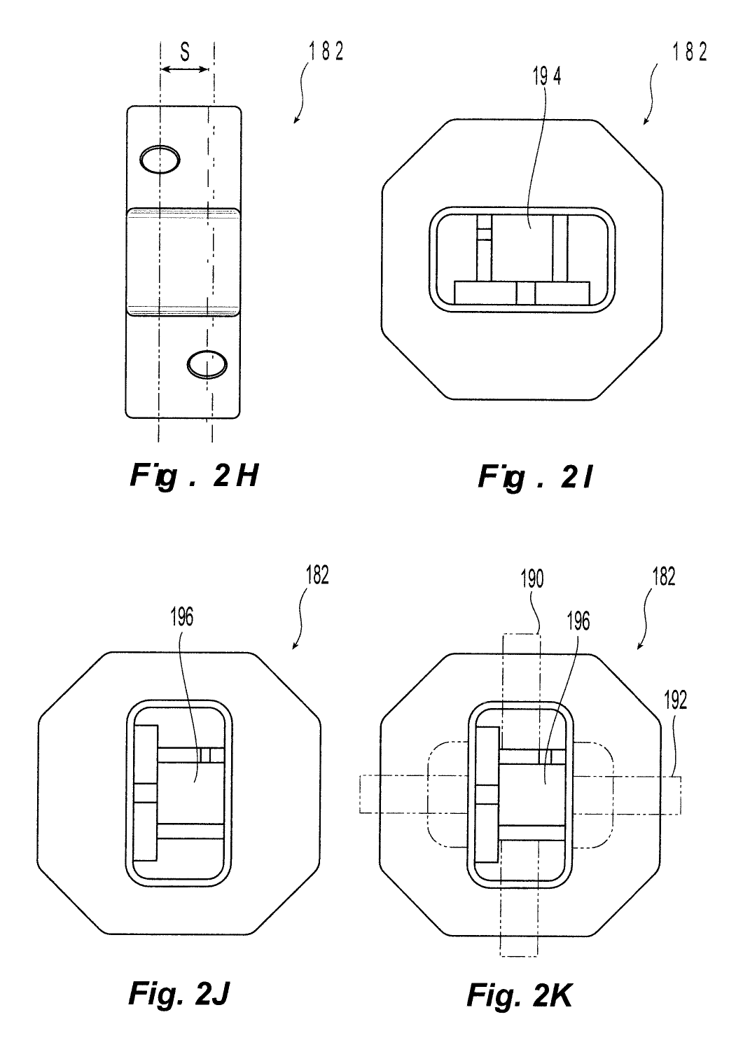

Turning to FIGS. 2A-2K, another exemplary embodiment of a high frequency ultrasound transducer holder 100 for providing an adjustable fluid interface in accordance with the present invention is shown. Holder 100 is substantially the same as holder 10 described above, and thus the descriptions of holder 10 with balloon 52 apply equally and apply equally to holder 100 with balloon 152.

Holder 100 differs from holder 10 in that positioning adjustment may be achieved in the X-Y plane using a fine adjustment system 180. While vertical adjustment screw 126 allows fine tuning of the focal zone depth associated with transducer 150 about or parallel to the Z-axis, fine adjustment system 180 provides additional positioning ability in the X-Y plane. In the exemplary embodiment shown, coupling 160 is fixed to fine adjustment system 180 instead of primary bracket 128 as was the case with respect to holder 10 and its primary bracket 28 and coupling 60.

As shown for example in FIG. 2C, fine adjustment system 180 includes a center block 182 disposed between a pair of plates 184, 186. Plate 184 is coupled to bottom ring 114 with spacers 188. Lead screws 190, 192 extend through block 182 and are spaced from one another in planes separated by a spacing s, as shown for example in FIGS. 2C and 2H. Lead screws 190, 192 are disposed perpendicular to one another to permit movement in X and Y directions. Lead screw 190 also may be received in plate extensions 184a, 184b of plate 184, while lead screw 190 also may be received in plate extensions 186a, 186b of plate 186. A first lead screw nut 194 is captured within block 182 and receives lead screw 190 while a second lead screw nut 196 is captured within block 182 and receives lead screw 192. Rotation of lead screws 190, 192 thus permits fine positioning of holder 100 in the X-Y plane. In a preferred exemplary embodiment, positioning adjustment may be achieved in the range of about ±1.5 inches in each of the X and Y directions.

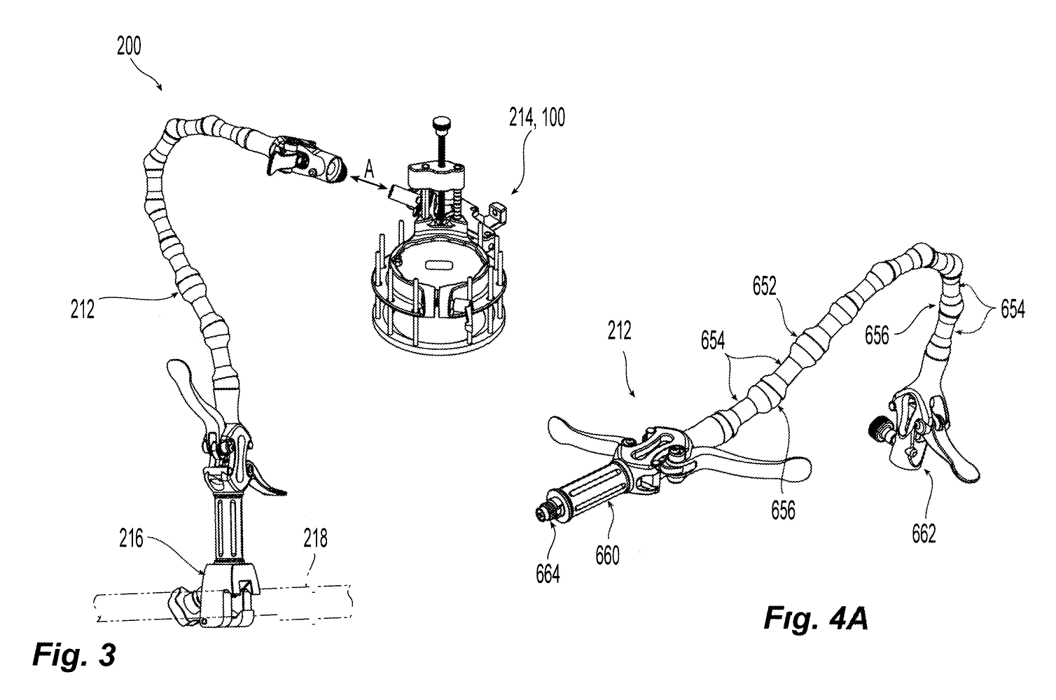

Referring to FIG. 3, an exemplary embodiment of a high frequency ultrasound transducer holder system 200 according to the present invention is shown. Holder system 200 includes a curvilinear articulating arm assembly 212 and a ultrasound transducer holder 214 coupled to assembly 212 as indicated by arrow A. As will be further described, arm assembly 212 includes a clamp 216 at a first free end thereof for coupling system 200 to a structure such as the rail 218 (shown schematically in phantom) of an operating room table.

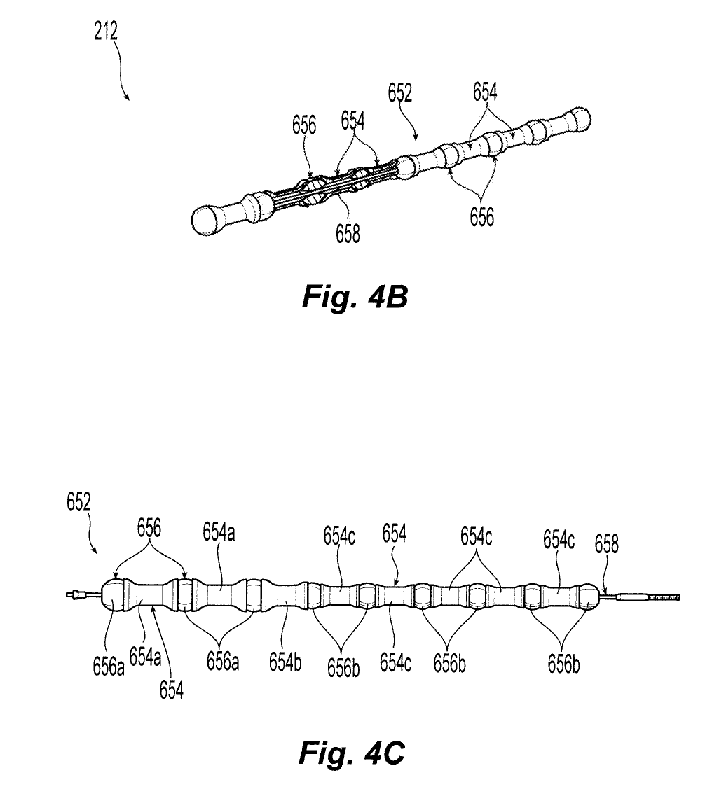

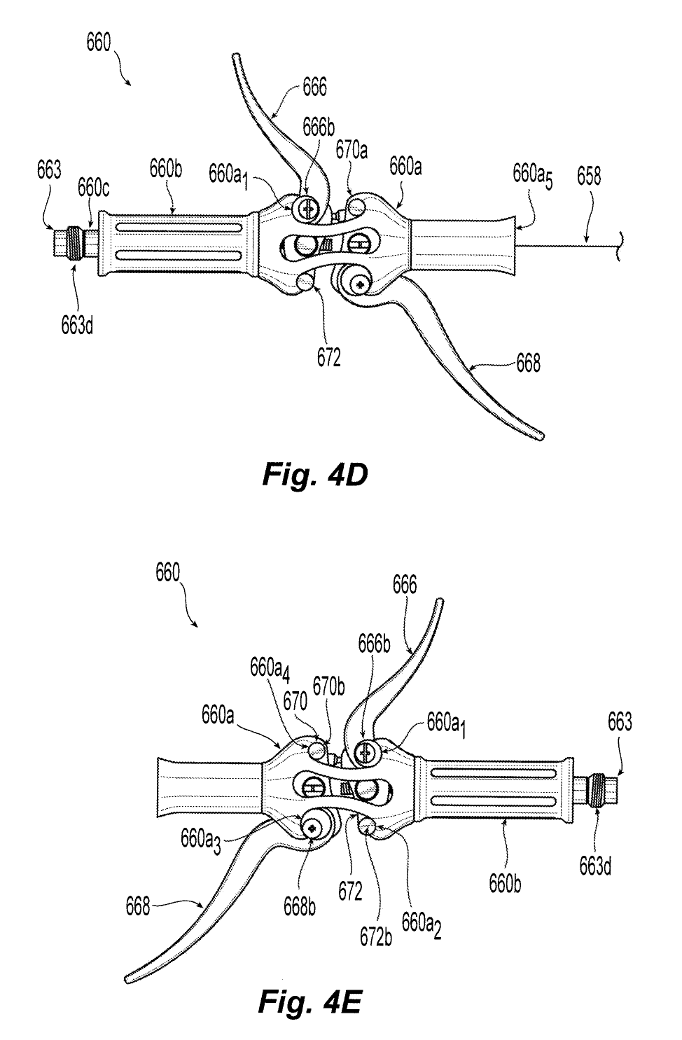

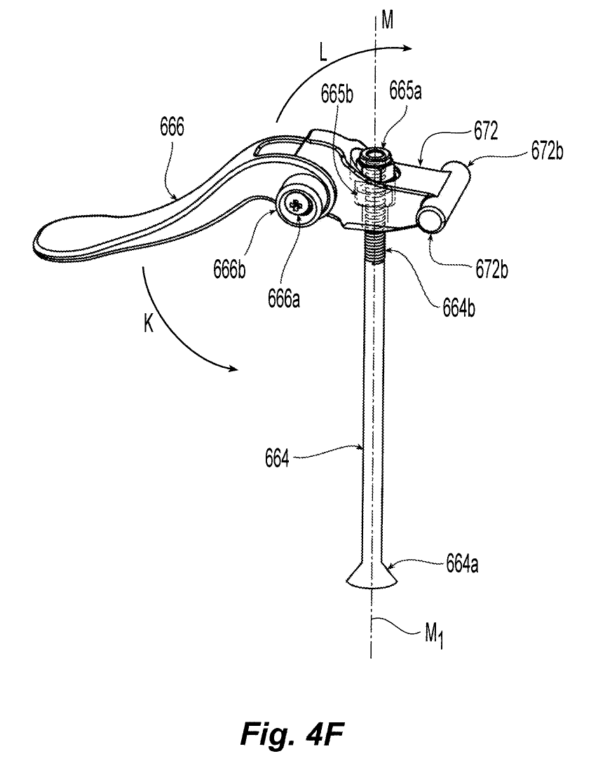

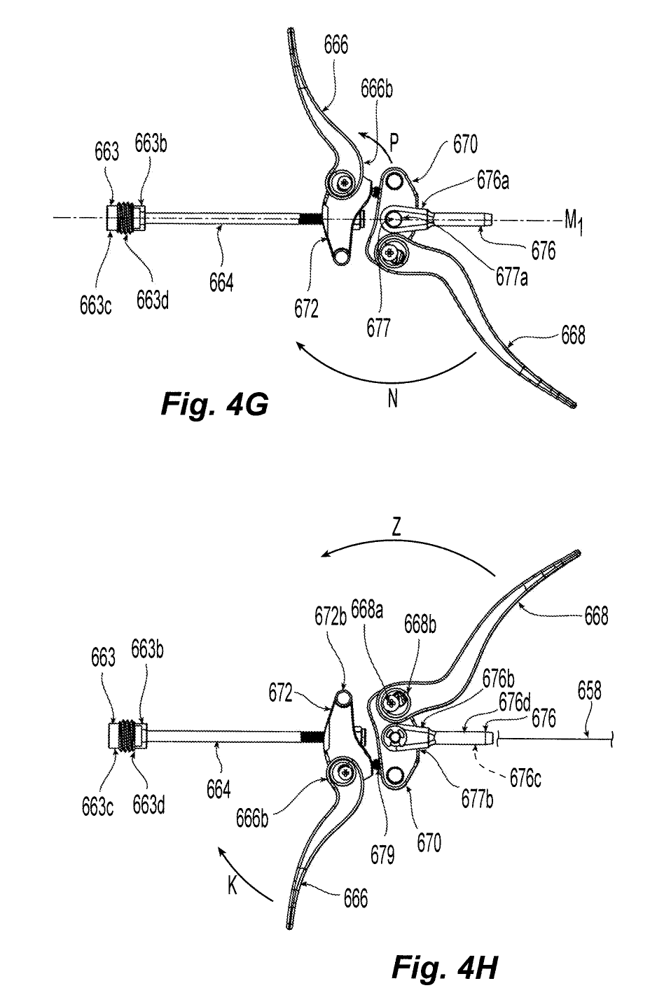

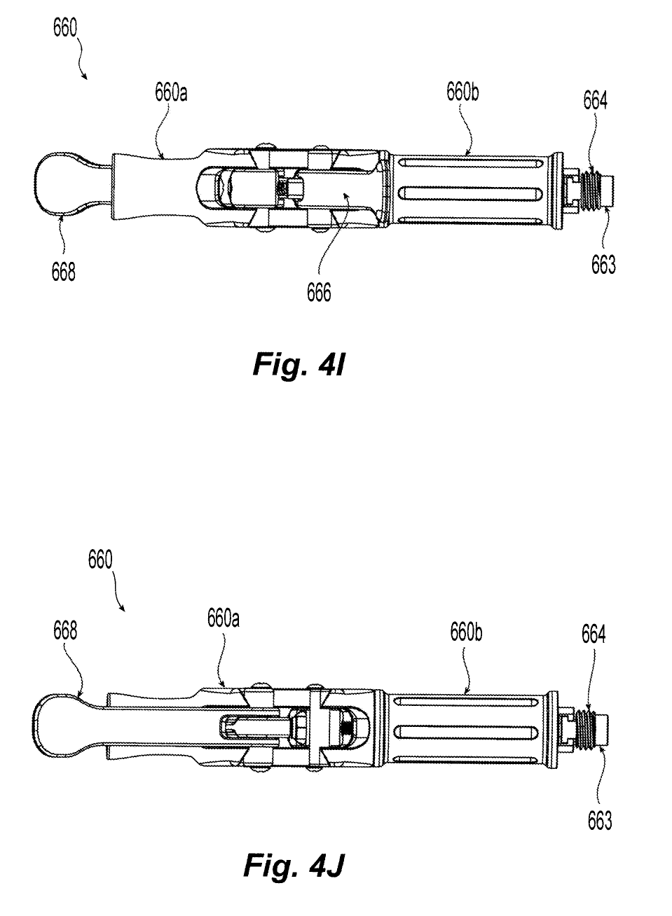

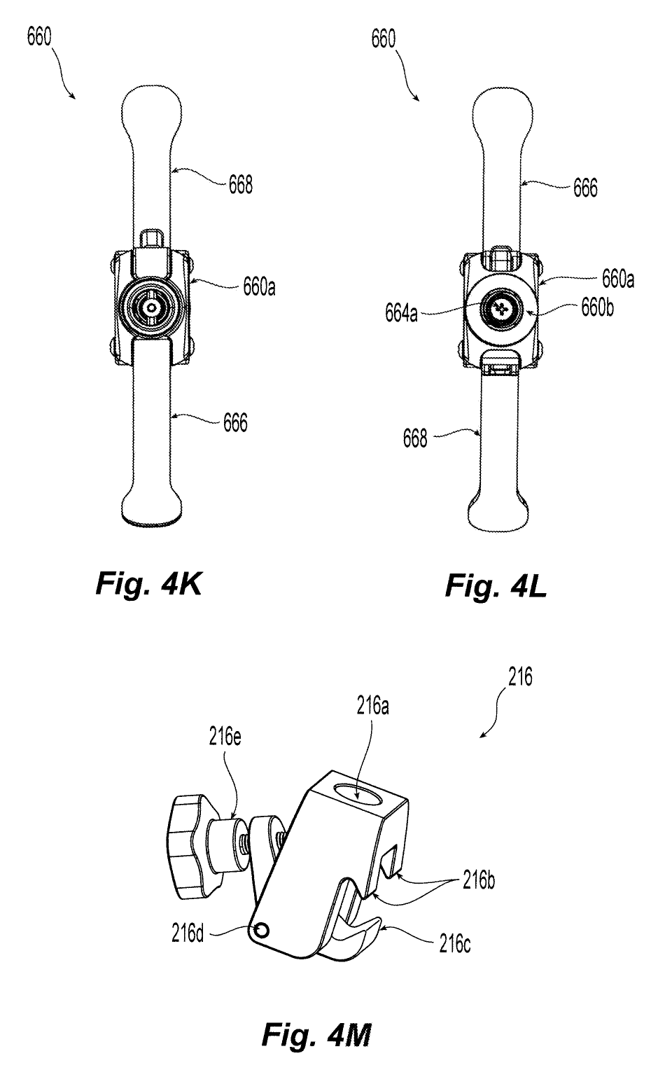

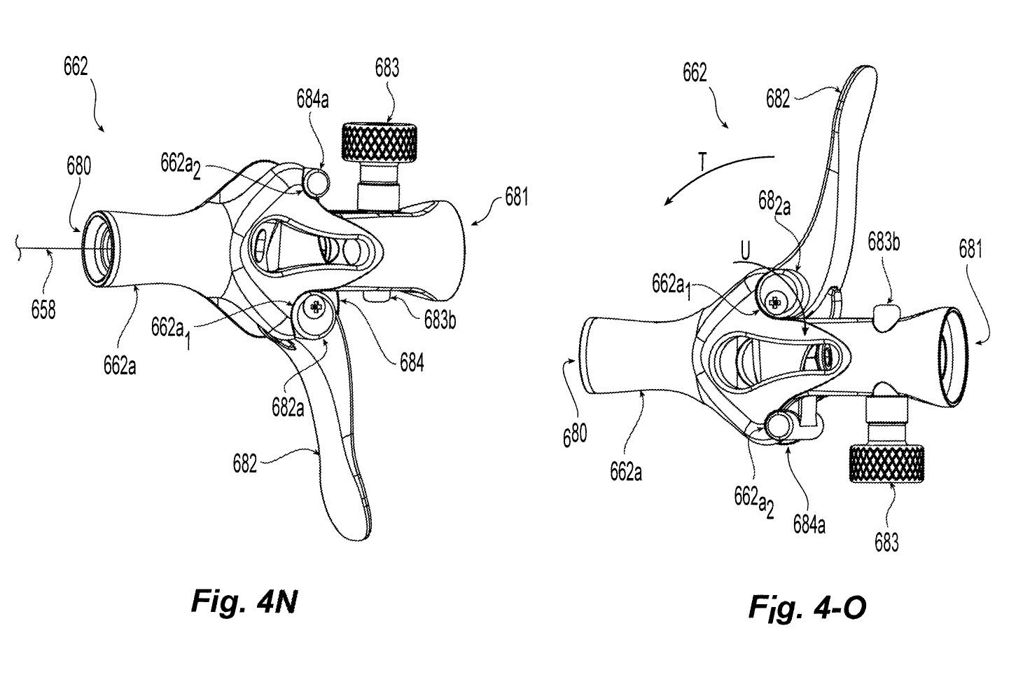

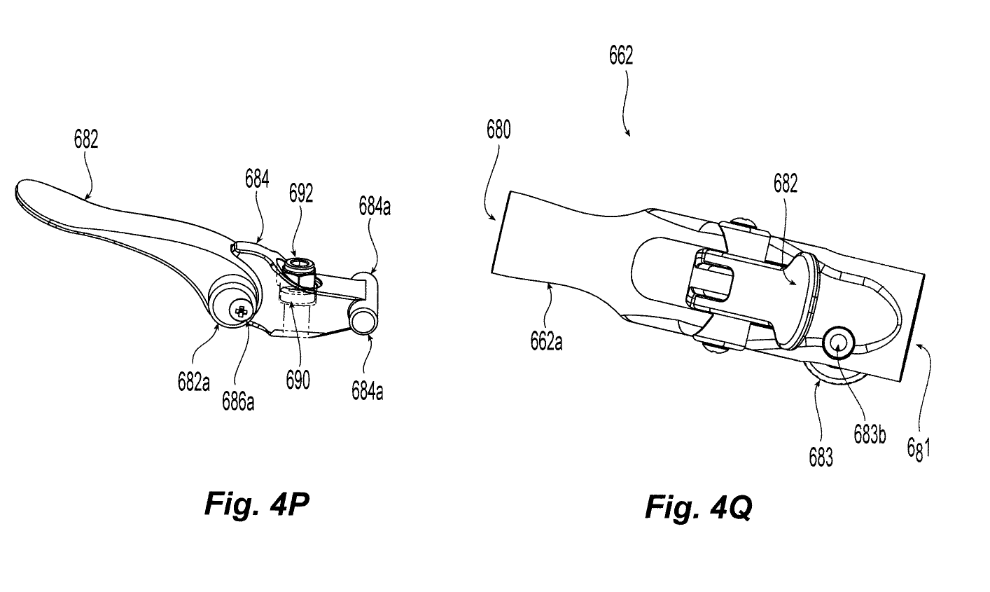

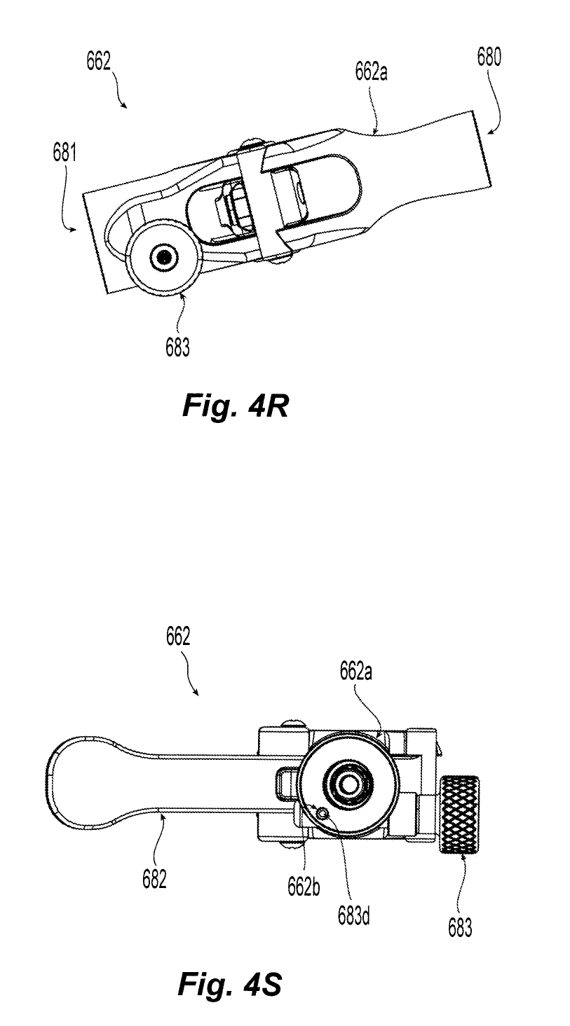

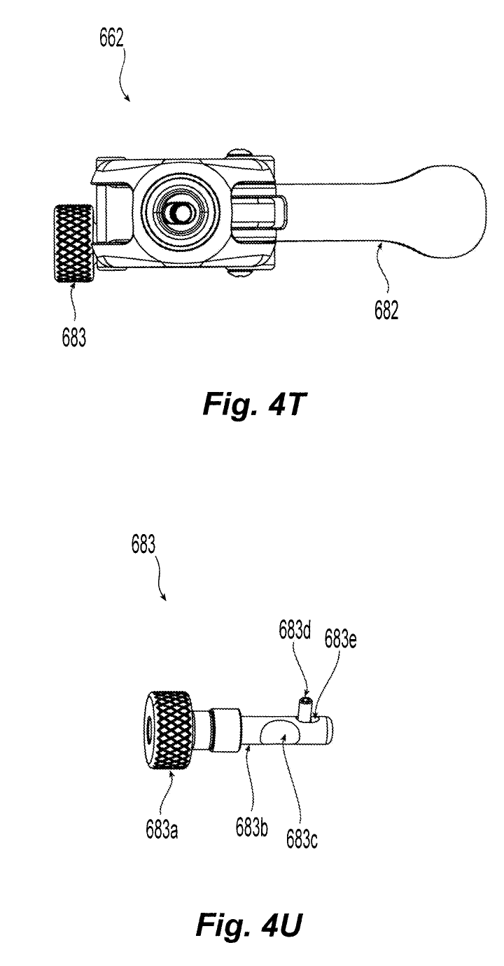

Turning to FIGS. 4A-4C, an exemplary preferred curvilinear articulating arm assembly 212 is shown for use with a transducer holder 214. Arm assembly includes a central arm 652 with a ball-sleeve arrangement that forms joints. In particular, central arm 652 includes a plurality of sleeves 654 with spherical balls 656 disposed therebetween thus forming ball and socket connections. In the exemplary embodiment shown in the figures, three balls 656a of a first size are disposed adjacent one another proximate one end of arm 652, while the remaining balls 656b are of a second size smaller than the first size. Sleeves 654a of a first size and sleeves 654c of a second size smaller than the first size are provided for accommodating balls 656a, 656b, respectively, while a transition sleeve 654b is provided intermediate sleeves 654a, 654c as shown for accommodating a ball 656a on one side and a ball 656b on the other side thereof. Sleeves 654 are configured and dimensioned to receive balls 656a, 656b at ends thereof and thus permit articulating of sleeves with respect to each other. A tensioning wire 658 runs generally centrally through sleeves 654 and balls 656, as will be further described shortly. Preferably, wire 658 is formed of metal. In an exemplary preferred embodiment, wire 658 is Type 302 stainless steel wire rope, 1×19 strand, 5/32 inch diameter, with a breaking strength of 3300 lb. (McMaster-Carr part number 3458T27). One exemplary operation of a wire tensioning mechanism is shown and described in U.S. Pat. No. 3,858,578 to Milo, which is expressly incorporated herein by reference thereto. Preferably, curvilinear articulating arm assembly 212 may move with six degrees of freedom.

In the exemplary preferred embodiment, three additional balls 656a and three additional sleeves 654a are provided to the arm assembly 212 shown in FIGS. 4A-4C, with arm assembly 212 having a fully extended (straightened) length of about 40 inches. In other embodiments, other desired lengths of arm assembly 212 may be accomplished by changing the number of balls and sleeves. For example, without the three additional balls 656a and three additional sleeves 654a, arm assembly 212 may have a length of about 32 inches.

A base handle 660 is coupled to central arm 652 on a first end thereof, preferably adjacent a ball 656a. In addition, a free handle 662 is coupled to central arm 652 on a second end thereof, preferably adjacent a ball 656b.

In one preferred exemplary embodiment, a series of larger balls 656a is provided proximate base handle 660 to provide stability to curvilinear articulating arm assembly 212. If for example a user such as a surgeon orients assembly 212 by grasping it proximate free handle 662, substantial bending forces may be exerted on central arm 652 proximate base handle 660. Thus, the use of larger balls 656a proximate base handle 660 as compared to smaller balls 656b proximate free handle 662 provides a system with larger surface area balls near base handle 660 for additional resistance to rotational movement in that portion of central arm 652 and thus more stability. In alternate embodiments, more than two different sizes of balls 656 or more than two sets of sizes of balls 656 may be used, preferably increasing in size toward base handle 660. In one alternate embodiment, each of the balls 656 in central arm 652 is of increasingly larger size from free handle 662 to base handle 660. The use of only two sizes of balls 656 advantageously facilitates manufacture and construction of arm assembly 212 because of the need to only stock two sizes as compared to a larger number of sizes and concomitantly greater ease of construction because only two sizes need be assembled to form central arm 652. In yet another alternate embodiment, central arm 652 may be formed of balls 656 that all are the same size.

Turning to FIG. 4D-4L, base handle 660 will be described. Base handle 660 includes a body portion 660a with levers 666, 668 pivotably associated therewith, as well as an extension 660b that turns screw coupling 663 and rotates in relation to and independent of body portion 660a. Base handle 660 further includes cam mechanisms 670, 672 as will be described. Portion 663b of coupling 663 preferably is noncircular and mechanically engages and is fixed to a like-shaped and sized non-circular opening in portion 660c of extension 660b so that rotation of extensions 660b as by gripping and turning by a user imparts like-rotation of coupling 663 for example for demountable coupling to clamp 16 and further coupling to a surgical table rail 18, as shown for example in FIG. 3. In the preferred exemplary embodiment, coupling 663 comprises a threaded portion 663d which may be threadably received in a threaded hole 16a disposed in clamp 16.

Coupling 663 is disposed proximate a first free end 664a of a stainless steel shaft 664 which extends therethrough and is provided with a head that abuts a shoulder disposed in end 663c of coupling 663. Preferably, rotation of coupling 663 is independent of rotation of shaft 664. Shaft 664 preferably extends through a hole in extension 660b.

Lever 666 is pivotably coupled to rocker arm 672 with a pin 666a that is disposed such that rotation of lever 666 results in eccentric movement of rocker arm 672. As shown for example in FIGS. 4D-4E, cylindrical projections 666b of lever 666 are received and rotate in arcuate cradle portions 660a1 of body portion 660a, while cylindrical projections 672b of rocker arm 672 are received and rotate in arcuate cradle portions 660a2 of body portion 660a. Rotation of lever 666 toward screw coupling 663 in direction K lifts pin 666a, and because rocker arm 672 rests on pin 666a, rocker arm 672 is rotated in direction L in an eccentric fashion.

As seen particularly in FIG. 4F, shaft 664 includes a threaded portion 664b the free end of which is threadably associated with a nut 665a. Shaft 664 extends through a hole in rocker arm 672 and an unthreaded insert 665b with a hole therein which assists in guiding travel of rod 664 along the longitudinal axis thereof. Pivoting of lever 666 in direction K causes rotation of rocker arm 672, and with shaft 664 coupled to nut 665a and nut 665a abutting insert 665b, rod 664 is translated in direction M.

When coupling 663 is threaded into a like threaded hole by rotation of extension 660b, arm assembly 212 is relatively loosely coupled by the connection of coupling 663 to the hole. To firmly couple arm assembly 212, lever 666 may be pivoted in direction K so that threaded portion 663d of coupling 663 also moves in direction M and bears against the threads of the hole in which it is received. The leverage created by even slight movement of the threads against the threaded holes, on the order of tens of thousandths of an inch, creates a wedging effect that strongly locks arm assembly 212 to the hole.

Lever 668 of base handle 660 also is pivotably coupled to a rocker arm 670 with a pin 668a that is disposed such that rotation of lever 668 results in eccentric movement of rocker arm 670. As shown for example in FIGS. 4D-4H, cylindrical projections 668b of lever 668 are received and rotate in arcuate cradle portions 660a3 of body portion 660a, while cylindrical projections 670b of rocker arm 670 are received and rotate in arcuate cradle portions 660a4 of body portion 660a. Rotation of lever 668 toward screw coupling 663 in direction N lifts pin 668a, and because rocker arm 670 rests on pin 668a, rocker arm 670 is rotated in direction P in an eccentric fashion.

A forked member 676, which for example may be formed of stainless steel, is coupled to rocker 670 and includes substantially parallel prongs 676a, 676b which mate with side walls of rocker 670 as shown. Rocker 670 is pivotably associated with forked member 676, with a shaft 677 extending through aligned holes in prongs 676a, 676b and rocker 670. Shaft 677 may be provided with a head 677a and an external retaining ring 677b secured in a shaft groove proximate an end opposite head 677a to retain forked member 676 in association therewith and thus with rocker 670. An axial through hole 676c is provided in tubular portion 676d of forked member 676. Tensioning wire 658 is coupled to forked member 676 by inserting an end portion of wire 658 in hole 676c and swaging tubular portion 676d so that wire 658, which extends out of open end 660a5 of body portion 660a, is retained by compression within tubular portion 676d.

When lever 668 is rotated in direction N, shaft 676 translates along the longitudinal axis M1 toward coupling 663 creating substantial tension in tensioning wire 658 such that movement of curvilinear articulating arm assembly 212 may be substantially resisted. In particular, actuation of second lever 668 may increase or decrease the tension in wire 658 as desired by acting on rocker arm 670. By increasing tension in wire 658, central arm 652 preferably becomes increasingly resistant to movement although central arm 652 preferably still may be moved through its full range of motion. Thus, a user may orient curvilinear articulating arm assembly 212 as desired, and then increase the tension of wire 658 so that the orientation of arm 652 is releasably fixed. Lever 668 preferably has an angular range of movement about pin 668a of up to about 180° to permit substantial tension to be generated in tensioning wire 658.

Rockers 670, 672 preferably are associated with each other as with a spring plunger 679 extending from within one rocker 670 into a hole in the other rocker 672. Spring plunger for example may be a stainless steel spring plunger with a round Delrin nose, without a lock element, with ¼″-20 threading, and 3-13 lb. end force (McMaster-Carr part number 84765A33). Spring plunger 679 is used as shown because under the force of gravity, first lever 666 may otherwise tend to move toward a closed position with in the direction of arrow K. Instead, spring plunger 679 applies pressure to rocker arm 672 to set lever 666 to tend to a default open position in which shaft 664 has not otherwise been raised toward open end 660a5 of body portion 660a.

In a preferred exemplary embodiment, rocker 670 moves with substantially greater eccentricity than rocker 672.

Clamp 216 for use with base handle 660 may be demountably attached to surgical table rail 218. As previously discussed, actuation of first lever 666 permits a user to apply a force on coupling 663 so that movement is resisted (e.g., in response to an 8 or 10 pound force applied to arm 652). In an alternate embodiment which will be further described later, screw coupling 664 as shown in FIG. 4A proximate base handle 660 of arm assembly 212 may be threadably associated with a threaded hole in another support surface.

A preferred exemplary embodiment of clamp 216 is shown in FIG. 4M. Clamp 216 includes a threaded hole 216a for threadably receiving threaded portion 664 of base handle 660. In addition, clamp 16 includes fixed jaw portion 216b and movable jaw portion 216c which is pivotable about axle 216d and lockable in place using screw mechanism 21 6e to firmly couple clamp 216 to a rail 218 secured between jaw portions 216b, 216c.

Next turning to FIGS. 4N-4U, free handle 662 will be described. Free handle 662 includes a wire receiving portion 680 and an end effector receiving portion 681. In particular, wire receiving portion 680 preferably is configured to receive a ball 656b therein, along with an end portion of wire 658. As described previously with respect to base handle 660, a pivotable lever 682 is associated with free handle 662 and preferably is coupled to tensioning wire 658 so that actuation of lever 682 may increase or decrease the tension in wire 658 as desired by acting on rocker arm 684. By increasing tension in wire 658, central arm 652 preferably becomes less flexible. Thus a user may orient curvilinear articulating arm assembly 212 as desired, and then increase the tension of wire 658 so that the orientation of arm 652 is releasably fixed. Free handle 662 has a body portion 662a, and lever 682 is rotatable with respect thereto. An interface lock 683 also is rotatably associated with body portion 662a proximate end effector receiving portion 681, as will be described shortly.

Lever 682 is pivotably coupled to rocker arm 684 with a pin 686a that is disposed such that rotation of lever 682 results in eccentric movement of rocker arm 684. Cylindrical projections 682a of lever 682 are received and rotate in arcuate cradle portions 662a1 of body portion 662a, while cylindrical projections 684a of rocker arm 684 are received and rotate in arcuate cradle portions 662a2 of body portion 662a. Rotation of lever 682 toward wire receiving portion 680 in direction T lifts pin 686a, and because rocker arm 684 rests on pin 686a, rocker arm 684 is rotated in direction U in an eccentric fashion.

Rocker arm 684 includes a hole in which a self-aligning setup washer 690 (a two-piece washer with one portion that rocks in another portion) is disposed. Setup washer 690 for example may be an 18-8 stainless steel self-aligning setup washer, ¼ inch in size, 17/64 inch inner diameter, ½ inch outer diameter, and 0.250 inch to 0.281 inch thick (McMaster-Carr part number 91944A028). A nut 692 also may abut setup washer 690 on the flat upper surface thereof and rock thereon. A threaded stud (not shown) may 30 be swaged to the end of tensioning wire 658 opposite the end attached to forked member 676, thus coupling wire 658 to the threaded stud by compression. The threaded stud may in turn be threadably associated with nut 692. Wire 658 is provided with suitable length to span from forked member 676 to nut 692.

Pivoting of lever 682 in direction T causes rotation of rocker arm 684, and with tensioning wire 658 coupled to nut 692 and nut 692 abutting insert 690, tension in wire 658 may be increased. In particular, actuation of lever 682 may increase or decrease the tension in wire 658 as desired. By increasing tension in wire 658, central arm 652 preferably becomes increasingly resistant to movement although central arm 652 preferably still may be moved through its full range of motion. Thus, a user may orient curvilinear articulating arm assembly 212 as desired, and then increase the tension of wire 658 so that the orientation of arm 652 is releasably fixed. Lever 668 preferably has an angular range of movement about pin 686a of up to about 90° to permit tension to be generated in tensioning wire 658.

In the preferred exemplary embodiment, actuation of lever 682 free handle 662 permits initial tensioning of central arm 652 while still permitting restricted movement. And, actuation of lever 668 of base handle 660 permits substantially greater tensioning of central arm 652 while also still permitting restricted movement thereof, Advantageously, with tension created in wire 658 of central arm 652 to restrict movement thereof, the orientation of lever 668 such as with respect to a patient still may readily be reset or adjusted before lever 666 in base handle 660 is actuated to create sufficient force to prevent rotation of threaded portion 663d of coupling 663 in the hole in which it is received.

As shown in FIG. 4U, interface lock 683 includes a knurled knob portion 683a and a cylindrical post 683b that is provided with an arcuate cutout 683c. Interface lock 683 is coupled to body portion 662a with set screw 683d which is threadably received in a threaded hole 662b in body portion 662a. Set screw 683d is further received in a slot 683e in post 683b to lock post 683b in a position with arcuate cutout 683c oriented to be movable along the longitudinal axis of cylindrical post 683b. Cylindrical post 683b may be disposed in a disengaged position in which the axial position of post 683b is such that arcuate cutout 683c generally follows the inner cylindrical contour of end effector receiving portion 681. Also, cylindrical post 683b may be disposed in an engaged position in which the axial position of post 683b is such that a portion of cylindrical post 683b other than arcuate cutout 683c extends past the inner cylindrical contour of end effector receiving portion 681 toward the central longitudinal axis of end effector receiving portion 681.

In use, in order for example to couple articulating arm assembly 212 to an end effector such as a holder 214, 100, by capturing post 102 of holder 100 in end effector receiving portion 681 of free handle 662, post 102 is inserted therein while interface lock 683 is disposed in the aforementioned disengaged position. While lock 683 is in the disengaged position, post 102 may freely rotate about the central axis of receiving portion 681. Once a desired orientation is set, lock 683 may be translated along the major axis defined by slot 683a so that a portion of cylindrical post 683b of lock 683 is disposed in an engaged position and bears against post 102. Such interference between post 102 of holder 100 and post 683b of lock 683 provides sufficient pressure so that post 102 will remain fixed in rotational position and translation along the longitudinal axis thereof against the inner cylindrical contour of end effector receiving portion 681.

Although an exemplary curvilinear articulating arm assembly is described herein, it should be understood that other preferably, curvilinear articulating arm assemblies instead may be used which preferably provide six degrees of freedom of movement and permit relatively rigid positioning such as described herein.

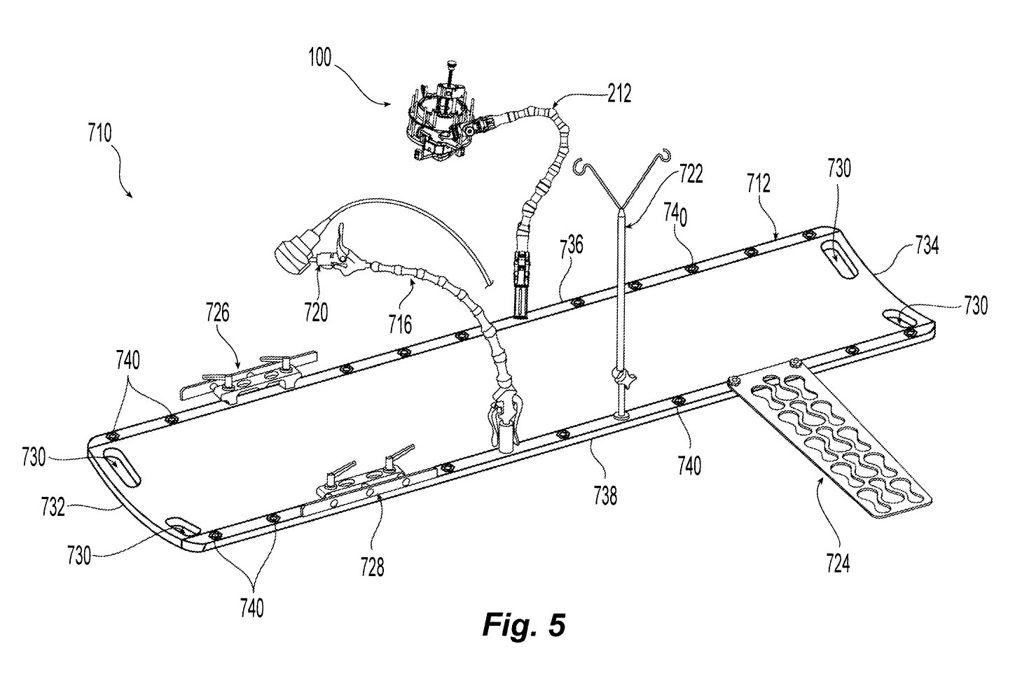

In some embodiments of the present invention, a transducer holder system such as system 200 may be coupled to a patient support other than a rail of a table. For example, referring next to FIG. 5, an exemplary support system 710 according to the present invention is shown with a variety of components coupled thereto. Support system 710 includes a tray 712, curvilinear articulating arm assemblies 212, 716 having respective end effectors 100, 720, an IV pole 722, an arm board 724, and rail assemblies 726, 728. A variety of end effectors may be demountably attached for example to articulating arm assembly 716 to assist a technician or practitioner with a medical/imaging procedure or provide other features useful with respect to a patient. End effector 720, for example, is configured as a self-centering abdominal probe bracket.

In one preferred exemplary embodiment, tray 712 may include two pairs of hold regions 730, each pair being disposed proximate a free cranial end 732 or free caudal end 734 of tray 712. In alternate embodiments, other numbers of hold regions 730 may be provided such as two or more, and hold regions 730 may be provided in other regions of tray 712 such as intermediate ends 732, 734 proximate sides 736, 738. Hold regions 730 may be configured as hand holds, or alternatively may be configured to receive strapping so that tray 712 may be releasably coupled to another object such as an ambulance stretcher, hospital bed, operating room table, or imaging scanner table. In some embodiments, handles may be coupled to tray 712. As also shown in FIG. 5, attachment regions 740 are provided proximate sides 736, 738 for demountably coupling components as previously described to tray 712, as will be further described below. In the exemplary preferred embodiment, tray 712 is provided with thirteen attachment regions 740, although in alternate embodiments another number of regions 740 may be provided such as at least one or tray 712 may be provided with a surgical rail or track permitting substantial freedom of coupling of components along the length thereof.

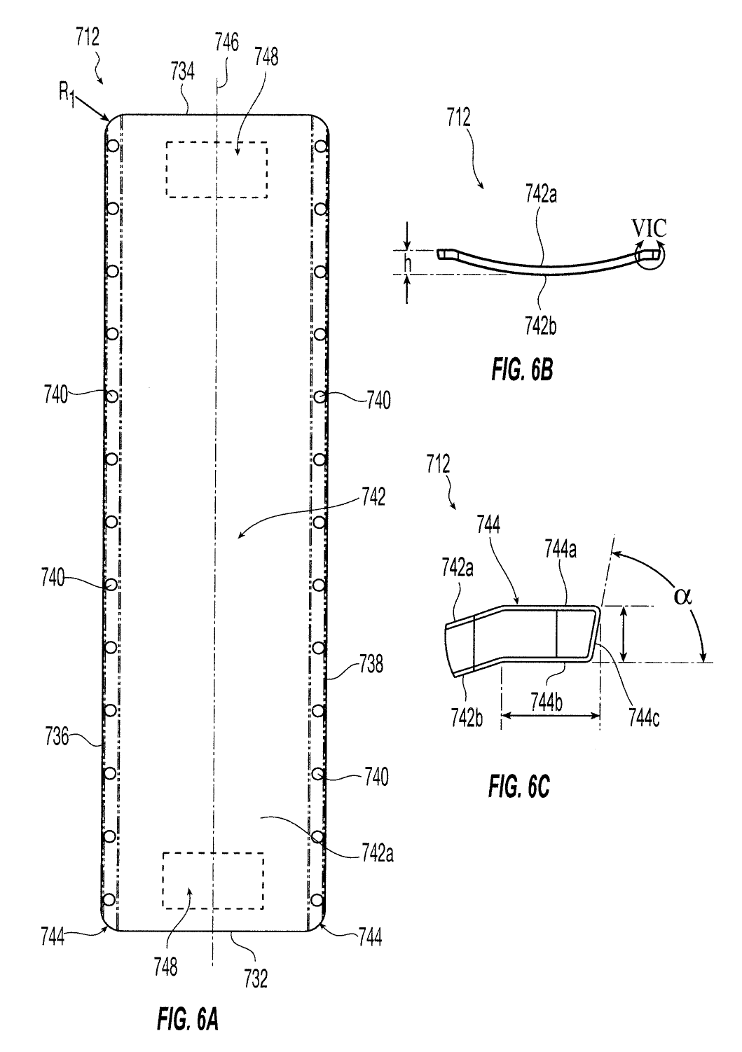

Turning to FIGS. 6A-6C, additional features of tray 712 are shown. Although hand hold regions 730 are not included in the figure, such regions may be provided as shown in FIG. 5. Attachment regions 740 are provided in spaced arrangement along the perimeter of tray 712. Preferably, tray 712 includes a central arcuate portion 742 disposed between outer ledge portions 744. Preferably, regions 740 are provided on outer ledge portions 744. Central arcuate portion 742 preferably has an upper concave surface 742a for receiving a patient and optionally a cushion (not shown) for the patient to rest against, and optionally includes a lower convex surface 742b. Preferably, outer ledge portions 744 include upper and lower surfaces 744a, 744b connected by a sidewall 744c at an angle α with respect to surface 744b. In a preferred exemplary embodiment, sidewall 744c is disposed at an angle a between about 60° and about 100°, more preferably between about 70° and about 90°, and most preferably at about 80°.

In a preferred exemplary embodiment, tray 712 is formed of natural finish carbon fiber, R-51 foam core, and phenolic. Attenuation preferably is less than 1 mm Al equivalency. Thus, tray 712 is radiolucent and suitable for use with computed axial tomography (CT) scanners. In other embodiments, tray 712 is formed of a material suitable for use with magnetic resonance imaging (MR) scanners. In addition, tray 712 preferably supports a load of 900 lbs. evenly distributed along centerline 746, about which tray 712 may be substantially symmetric as shown. Indicia 748 optionally may be provided, as shown for example proximate ends 732, 734. The indicia may for example indicate preferred orientation of tray 712 with respect to a patient lying thereon.

In the preferred exemplary embodiment, attachment regions 740 on each side of tray 712 are evenly spaced from each other by about 6 inches between centers thereof. To accommodate patients and equipment attached to tray 12, in one preferred embodiment tray 712 has a length of about 78 inches, a width of about 21 inches, a generally uniform thickness of about 0.9 inch, and a height h of about 2.5 inches. Comers may be provided with a radius R1 of about 2 inches. In the preferred exemplary embodiment, attachment regions 740 preferably accommodate threaded inserts, which may be formed of aluminum.

In some embodiments, tray 712 is sized to hold an adult patient, and may be between about 180 cm and about 200 cm long. However, it will be appreciated that longer and shorter trays may be provided. In order to accommodate an adult patient, tray 712 may support an overall weight capacity of at least about 200 pounds, and preferably at least about 300 pounds. However, if a tray 712 is sized for use with a pediatric patient, tray 712 may only accommodate weights that do not exceed 200 pounds, and more preferably do not exceed 100 pounds.

Although the surface of portion 742 of tray 712 is substantially smooth in the preferred exemplary embodiment, in alternate embodiments the surface may be textured to provide additional resistance to motion of objects and/or a patient placed thereon.

Tray 712 thus is suitable for use in multiple environments, and thus may “move” with the patient from one environment (e.g., ambulance) to the next (e.g., CT scanner) without removing a patient supported thereon.

While various descriptions of the present invention are described above, it should be understood that the various features can be used singly or in any combination thereof. Therefore, this invention is not to be limited to only the specifically preferred embodiments depicted herein.

Further, it should be understood that variations and modifications within the spirit and scope of the invention may occur to those skilled in the art to which the invention pertains. For example, although a balloon 52 is disclosed herein for filling with a fluid interface medium such as degassed water to permit energy transmission, other types of fluid reservoirs and configurations may be used. Furthermore, any of a variety of couplings may be used to releasably attach holders 10, 100, 214 as disclosed herein to an articulating arm; such couplings should provide secure attachment. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

What is claimed is:

1. An ultrasound transducer positioning system comprising:
a curvilinear articulating arm;
an ultrasound transducer; and
a holder comprising a first region for receiving the transducer and a second region for a fluid interface disposed adjacent the first region, the holder being coupled to the curvilinear articulating arm;
wherein the second region is adjustable in size.

2. The system of claim 1, wherein the first region is defined by a generally circular clamp.

3. The system of claim 2, wherein the clamp comprises a split ring.

4. The system of claim 2, farther comprising a lead screw coupled to the clamp.

5. The system of claim 4, wherein:
the second region is defined between the clamp and an opposing member; and
the lead screw extends to the opposing member.

6. The system of claim 5, wherein the opposing member comprises a ring.

7. The system of claim 2, wherein the transducer is retained by at least one member coupled to the clamp and abutting a portion of the transducer.

8. The system of claim 7, wherein the at least one member abuts a portion of a face of the transducer.

9. The system of claim 1, wherein the fluid interface is a flexible member.

10. The system of claim 1, further comprising at least one rail with indexing thereon, the rail extending between a bracket coupled to the curvilinear articulating arm and a clamp defining the first region.
